(19) United States
(12) Patent Application Publication
(10) Pub. No.: US 2024/0032864 A1
(43) Pub. Date: Feb. 1, 2024

(54) BIOMETRIC DATA COLLECTION AND DISPLAY SYSTEMS

(71) Applicant: StratoScientific, Inc., Shoreline, WA (US)

(72) Inventors: Mahesh S. Mulumudi, Snohomish, WA (US); Craig S. Rosenberg, Shoreline, WA (US)

(21) Appl. No.: 18/359,839

(22) Filed: Jul. 26, 2023

Related U.S. Application Data
(60) Provisional application No. 63/392,626, filed on Jul. 27, 2022.

(51) Int. Cl. A61B 5/00; A61B 5/256; A61B 5/0205

(52) U.S. Cl. CPC A61B 5/6822; A61B 5/256; A61B 5/0205; A61B 5/742

(57) ABSTRACT

The arrangements of the present disclosure relate to a device for detecting biometric data, including a necklace having a necklace body configured to be worn around a user's neck, a plurality of sensors disposed on the necklace body, for detecting biometric data of the user when the necklace is worn around the user's neck, at least one of an electronic transmitter for transmitting electronic signals corresponding to the biometric data, or an electronic memory for storing biometric data.

### CROSS-REFERENCE TO RELATED PATENT APPLICATION

This application claims priority under 35 U.S.C. § 119(e) to U.S. Provisional Application No. 63/392,626, filed Jul. 27, 2022, the contents of which are incorporated herein by reference in its entirety.

### BACKGROUND

The disclosed technology is related to the field of collection and/or monitoring of the biological signals as a wearable device that is worn around the neck, and it also relates to the field of health monitoring for either continuous or episodic collection and/or monitoring of biological signals for healthcare evaluation.

### SUMMARY OF THE INVENTION

The disclosed technology herein also relates to creating visual and audio representations of bio signals for purposes of medical evaluation, mindfulness enhancement, therapy, and entertainment purposes.

In light of the foregoing disadvantages of the prior art, it is an object of embodiments of the present disclosed technology to provide a necklace to detect the aforementioned biological signals when worn, without the use of hands, arms, or legs.

It is a further object of the disclosed technology to detect acoustic signals using sensors or collectors such as, but not limited to one or more contact or acoustic microphones attached to or embedded in the body of the necklace at different strategic locations to target and receive biological signals from the desired organs.

It is a further object of the disclosed technology to detect electrical biological signals (for example, but not limited to, electrocardiogram, electromyogram, electroencephalogram) using contact leads embedded in the body of the necklace and/or an ornament attached to the necklace at one or more locations and separated by appropriate distances.

It is a further object of the disclosed technology to detect optical or image signals representing biological information, with sensors embedded in the body of the necklace and/or the body of an ornament attached to the necklace at different strategic locations.

It is a further object of the disclosed technology to detect the aforementioned biological signals using appropriate collectors attached to or embedded in a pendant or multiple pendants attached to the necklace.

It is a further object of the disclosed technology to process biological information and signals provided by one or more electrocardiogram, electromyogram, electroencephalogram or other sensors with processing electronics attached to or embedded in the body of the necklace and/or the body of the ornament.

It is further object of the disclosed technology to have an integrated power supply like a rechargeable or disposable battery attached to or embedded in the body of the necklace and/or the body of the ornament.

It is a further object of the disclosed technology to have a power supply using a cord to supply such power.

It is a further object of the disclosed technology to make the neck sensors to be worn as a jewelry or a fashion accessory for decoration, ease of use, style, and personal satisfaction.

It is a further object of the disclosed technology to detect the movement of the necklace or the pendant with the use of sensors attached to or embedded in the body of the necklace and/or an ornament attached to the necklace at one or more locations and separated by appropriate distances.

It is a further object of the disclosed technology to have one or more biological signals detected from the necklace or the pendant to be modified by the system or the user to see its influence on the other biological signals.

It is a further object of the disclosed technology for the detected biological signals to be either independently or dependently associated with imagery on the computerized displays.

It is a further object of the disclosed technology for the signals from the necklace to be associated with imagery, sound, or other forms of representation to facilitate a user experience.

It is a further object of the disclosed technology to detect, record and/or monitor the biological signals and/or transmit them to either a handheld mobile computing device, a bodily worn computing device, an implanted computing device, or an external stationary computing device either wirelessly or with a wired connection that stores the signals, processes the signals, and displays the signals for a desired purpose.

### BRIEF DESCRIPTION OF THE DRAWINGS

FIG. 1 illustrates an example biometric necklace, according to various arrangements.

FIG. 2 illustrates an example biometric necklace, according to various arrangements.

FIG. 3 illustrates an example biometric necklace, according to various arrangements.

FIG. 4 illustrates an example biometric necklace, according to various arrangements.

FIG. 5, an example screenshot of an audio-visual program interfacing with a biometric necklace or another biometric data collection device, according to various arrangements.

### DETAILED DESCRIPTION

Collection of the biological signals like pulse, heart rate, temperature, electrocardiogram (EKG), electromyogram (EMG), electroencephalogram (EEG), and pulse oximeter readings can be achieved by various wearable devices on the wrist and attached to the chest. Such devices are used for the collection of biological signals and transmitted or processed by software elements either embedded in the device or on connected devices. There are disadvantages in using the existing disclosed technologies to collect the aforementioned biologicals. The most significant being the inability to collect the signals without the use of one's hands. Also, to collect electrical signals like an electrocardiogram, two or more independent electrical contacts separated by a given distance are used making the device cumbersome to comfortably fit on an appendage, like the arm. To overcome these disadvantages, we proposed a novel method to collect acoustic, electrical, optical, and magnetic biological signals without the use of the hands, arms, or legs.

Disclosed herein is a wearable necklace with or without a pendant having an ability to collect biological acoustic, electrical, optical and magnetic signals, to be utilized for collecting, monitoring, storing, processing or displaying, biological data from the individual wearing the necklace. The necklace has the necessary electrical contacts and/or sensors embedded into either the body of the necklace and/or an ornament attached to the necklace in order to collect and/or monitor biological signals. These collected and/or monitored signals can ben be transmitted either wirelessly or with a wired connection for either continuous or episodic monitoring of the bodily and mind functions. The necklace and/or the pendant that is attached to the necklace can be either powered by batteries, a cord, and/or in a cordless manner. The necklace can take many forms and is not limited to a string of beads, a cord, a chain, a rope, etc. The shape of the necklace may take the form of many different shapes or combinations of shapes and is not limited to specific shapes such as triangles, squares, rectangles, ovals, polygons, etc. The materials of the necklace may be composed of one or more materials and is not limited to metals, composites, organic materials, manufactured materials, etc. The size of the necklace may include a very wide range of sizes and is not limited in the length of the necklace, the diameter (or diameters) of the necklace, the weight or density of the necklace, etc.

Also disclosed herein is an graphical interface for rendering moving images (and optionally sound) based on biometric readings such as heart rate and breathing rate, wherein the user's biometrics control the movement of images in a generated visual scene observed by the user. For example, a user watching a screen or wearing virtual reality goggles can observe a nature scene wherein a wind appears to move objects back and forth in correspondence to the user's breathing, and another object (e.g., an animal) engaging in rhythmic movements corresponding to the user's heart rate. Such biometric feedback imagery is useful for mindfulness and meditation, as well as more generally visually representing a person's body functions for any number of purposes.

In some examples, the biometric readings used to render the moving images in the graphical interface are provided by a wearable device, such as, but not limited to the wearable necklace described herein. In other examples, the biometric readings are provided by one or more other biometric sensors.

Biometric Necklace

The objectives of certain embodiments of the disclosed technology are achieved by a necklace and/or a pendant attached to a necklace with embedded sensors to collect various biological signals at one or more locations that can be worn around the neck in a hands-free manner or by using a pendant attached to the necklace. The necklace (or its pendant) may be designed to cross near the user's trachea, or the user's chest, or the user's abdomen. Soft elastic bands or the like may be employed as needed to wrap around the user to secure the necklace and/or pendant in a desired position if might not otherwise tend to remain there. The sensor and the associated modules are located with adequate distance between them and oriented along the electrical axis of the organ being monitored.

For example, an electrocardiogram to monitor the heart would need a minimum of two electrical lead sensor modules, each pair located on either limb of the necklace so that the orientation would be along the vertical electrical axis of the user's heart, or on the same limb of the necklace, or oriented on the back of the neck and the front of the user's chest along the horizontal electrical axis of the heart, when the necklace is worn by the user.

For electromyogram (EMG) there need to be three electrode sensors that would be in contact with the group of muscle being monitored for activity, for example the muscle groups in the neck. In one example of the embodiment a muscle sensor module with one of the leads to be in contact with the end of the muscle insertion and the other in contact with the middle of the muscle bundle of the neck and a third electrode for reference would be on the chest with detect the muscle tension activity in the neck muscles.

For pulse oximetry, the optical sensor module may be in the limbs or the pendant of the necklace to be in contact with the skin on the back of the user's neck or the user's chest wall, when the necklace is worn by the user.

For EEG, sensor module may be located on the necklace, adjacent the back of the user's neck and the user's head for optimal acquisition of the biopotentials, when the necklace is worn by the user.

Examples of sensor modules that may be used include (a) AD8232 by Analog Devices Inc. for EKG and EMG signal amplification, (b) BR8 for EEG signal amplification, and (c) MAX30100 by Maxim Integrated for pulse oximetry signal amplification. The output from these sensor modules will be directed to an analog to digital convertor (such as, for example, the ADS1298 by Texas Instruments) housed in the pendant or the limbs of the necklace itself along with the power supply and further processed by the microcontroller unit (such as, for example, the MSP430F5522 by Texas Instruments) to function.

In one example, for collection of the acoustic signals a micro-electromechanical systems microphone or a contact microphone will be placed to be in proximity or contact with the neck for sounds from the trachea or the carotid arteries and over the upper or lower chest to collect sounds from the lungs and cardiac sounds. In one example, a TDK InvenSense, MMICT3902-00-12 (TDK corporation of America) may be utilized and embedded in a location to be in contact with or close to the neck and another in the upper and lower chest wall to collect sounds from the trachea and the lungs or heart respectively.

In some examples, the necklace and/or pendant may have processing electronics for processing electrical signals provided by a microphone or other sensor. The processing electronics may include one or more electronic processors, microprocessors, or the like. In some examples, the necklace and/or pendant may have a transmitter or other transmitting electronics, for wireless transmission of signals generated with the processor to one or more external devices as described herein.

In some examples, the necklace and/or pendant may have one or more non-transient electronic memory devices for storing signals or data associated with signals provided by the microphone or other sensors. The non-transient memory device may include a random access memory (RAM) or other suitable memory device.

The necklace and/or pendant with one or more sensors and processors may use either an embedded power supply using rechargeable or disposable batteries, or a wireless power connection combined with the necessary software that will allow it to connect with one or more computing devices that is either mobile, implantable, or wearable elsewhere on the body, inside the body, or external to the body. The connection to one or more computing devices can be achieved either using wireless protocols or via a wired connection.

It is advantageous to electrically isolate the individual sensors from each other as necessary to reduce or eliminate electrical interference, and to improve the signal to noise ratio of the biological signals and biopotentials being collected by the device. Towards this goal in one embodiment the necklace may have elements made of electrically inert material like silicone, plastic, or wood in which the sensors may be embedded. The electrical connection to and from the sensors to the digital converters and power supply may be in the form of an electrical conductor running between the beads of the necklace. The wireless communication modules that allow communication between the necklace and the target computing device can also be embedded in the beads of the necklace or its pendant.

Silicone may be used in constructing the body of the necklace, both because of its electrical insulating properties and because it can be designed to have a certain degree of friction with or “stickiness” to human skin that can help hold the necklace in a desirable position. Soft elastic bands can also used to secure the necklace and/or pendant as desired, wrapped around the user's body and connected to the device. If a more precise and controlled placement is desired, medical or athletic adhesive tape can be used to secure all or portions of the device to the user's skin. Additionally or alternatively, the necklace may be weighted so as to tend to stay in contact with the wearer's skin, and rigid or semi-rigid underwire can be used to help hold the necklace in a desired shape and in contact with the user's skin. In certain embodiments, the necklace may gently squeeze the back of the user's neck to help hold it in place and against the skin.

Other kinds of sensors may be added to the necklace to increase the kinds of data that may be collected. These may include gyroscopic or accelerometer sensors to detect user movements, or thermometers or thermocouples to detect temperature.

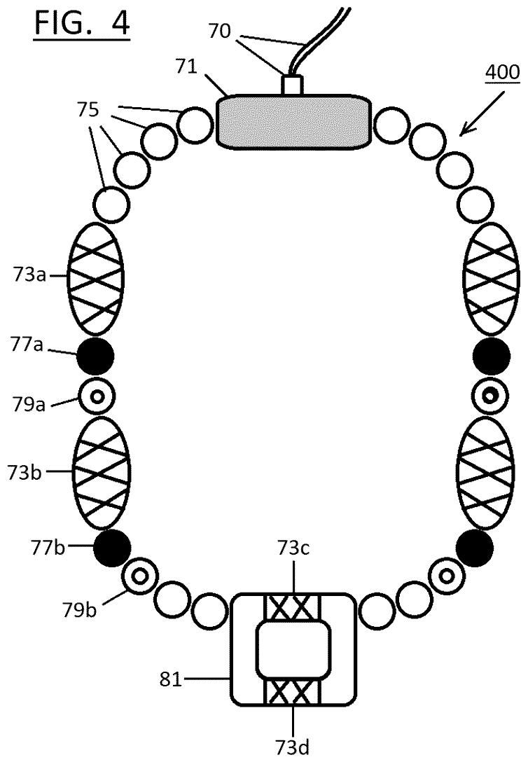

The body of the necklace may also conceal the electrical wiring connecting the various components, battery, and processors. This may be accomplished, for example, by having the wires encased in a hollow silicone tubing. This may also be accomplished by having the necklace comprise a series of structures (such as, for example, in FIG. 4) that are adjacent to each other and have the wiring pass through and between them through holes in those structures.

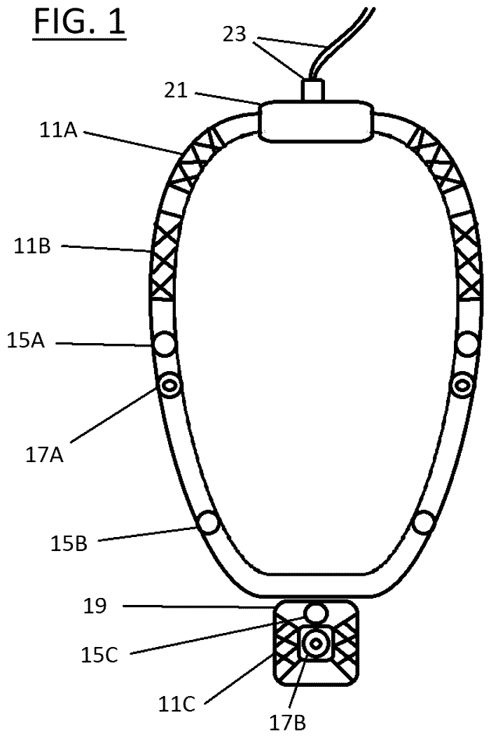

Referring to FIG. 1, an embodiment of a biometric necklace 100 is shown having three kinds of sensors dispersed around its periphery: electrical contacts 11A-C (as may be used to take EKG readings or the like), microphones 15A-C (for listening to body sounds), and optical sensors 17A-B for gathering visual and/or non-visual spectrum signals. (Note that equivalent structures are located on the opposing side of the necklace 100, if not specifically labeled.)

A housing 21 contains a microprocessor and a battery that supplies power to the necklace's accessories via wires (not shown) embedded in the necklace. The housing 21 may also serve as a weight to help hold the necklace generally against the skin. A power supply 23 may include charging electronics that can be selectively plugged into housing 21 to charge the battery. A weighted pendant 19 helps to hold the necklace 100 in contact with the user's skin when worn. A rigid or semi-rigid wire within the necklace (not shown) may be employed to help hold the necklace generally in a certain shape when worn, further improving skin contact and maintaining certain distances among the components.

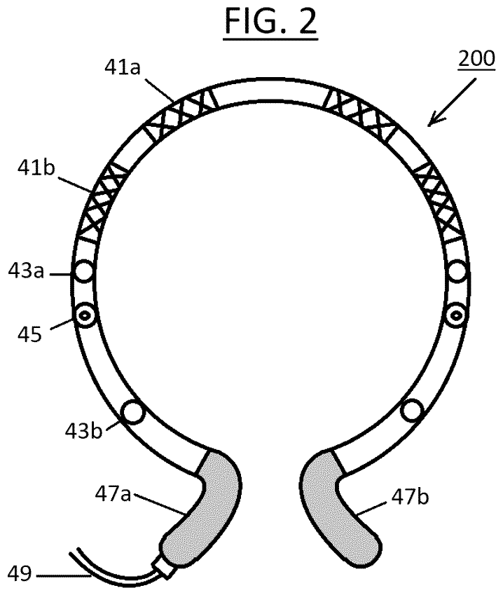

FIG. 2 depicts another example embodiment of a biometric necklace 200. The same types of components and features may be employed as in necklace 100, including electrical contacts 41a-b, microphones 43a-b, and optical sensors 45. The necklace 200 may have a semi-rigid wire within it (not shown) that allows the necklace to spread open to be placed around the user's neck, and then elastically re-tightening about the neck and making contact with the skin. A battery 47a and an equally-weighted microprocessor housing 47b are placed at the forward tips of the necklace and help to weigh the necklace against the skin. A power supply 49 is also shown.

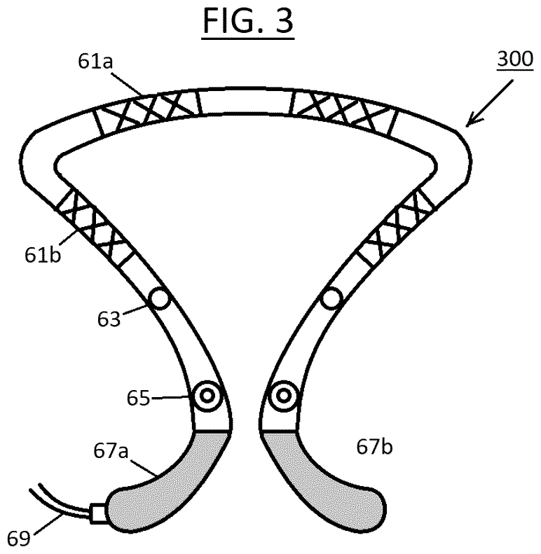

FIG. 3 depicts another example embodiment of a biometric necklace 300. The same types of components and features may be employed as in necklace 100, including electrical contacts 61a-b, microphones 63, and optical sensors 65. The necklace 300 may have a semi-rigid wire within it (not shown) that allows the necklace to spread open to be placed around the user's neck, and then elastically re-tightening about the neck and making contact with the skin. A battery 67a and an equally weighted microprocessor housing 67b are placed at the forward tips of the necklace and help to weigh the necklace against the skin. A power supply 69 is also shown.

FIG. 4 depicts another example embodiment of a biometric necklace 400. Beads 75 provide sufficient weight to help hold the electrical contacts 73a-d against the user's skin. Optical sensors 79a-b and microphones 77a-b are also employed, along with pendent 81. A housing 71 containing a microprocessor and a batter with power supply 70 is also shown.

Biometric Audio-Visual Feedback Scenes

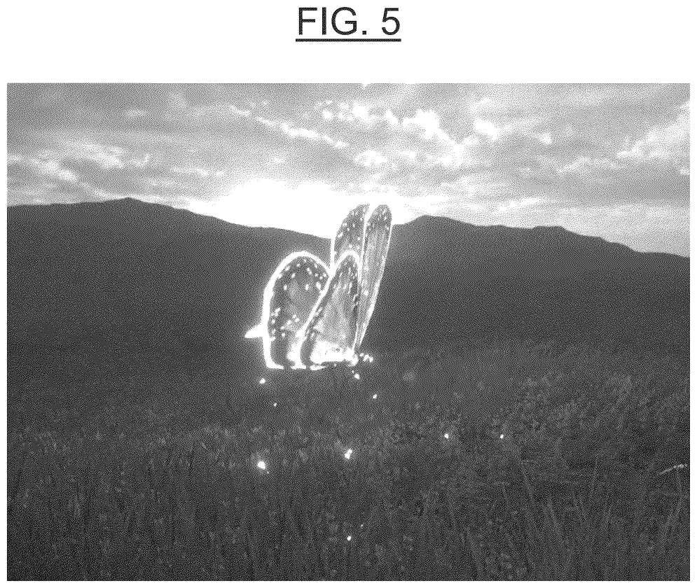

Referring to FIG. 5, an example screenshot is shown of an audio-visual program developed to interface with the disclosed biometric necklace, or with any other kind of biometric data collection device. In the example shown, a dynamically moving nature scene is generated in a virtual reality environment that can be seen by a user wearing a VR headset or watching a screen whose perspective changes based on physical orientation.

User biometric signals or data is fed into the computer system that controls the visual display, allowing live real-time reporting of biometric data. “Real-time” or “live” as used herein refers to data that is used or displayed at the same time as it is being collected (or nearly so, accounting for any lag-time), so that a user may see information or depictions of their biometric readings at the same time as they are happening. In some examples, the biometric signals or data is provided by a wearable device, such as, but not limited to the wearable necklace described herein. In other examples, the biometric signals or data are provided by one or more other biometric sensors.

The scene includes a wind that appears to move the tall grass on the ground back and forth to match the user's breathing rate, while butterflies have wings that flap in time to the user's heartrate, glowing brighter the more that the user is able to slow their heartrate, and releasing colorful lights as the user approaches a desired heartrate. Audio overlay can add the sound of breathing (or some other substitute sound at the same intervals), as well as the sound of the heartbeat (or some other substitute sound at the same intervals). An EKG or other electrical impulse signal can be represented in the scene as, for example, lighting in clouds that strikes and dissipates in a pattern corresponding the EKG readings.

The technology can be implemented with a wide variety of audio-visual scenes in which the movement and sounds of objects in the environment is dictated by the biometrics of a user, such as their heartrate, breathing rate (and intensity/volume), EKG and other electrical readings, blood pressure, and the like. While such scenes could be presented on a fixed two-dimensional screen (like a television or desktop computer monitor), the scenes will be more engaging if rendered as an immersive three-dimensional environment that can be experienced using virtual reality, or with a viewing device that otherwise can be moved in three-dimensional space to see other areas of the scene. An object of certain embodiments of the technology can be to create an artificial environment that allows a user to immerse themselves in their own biological feedback.

Purely abstract scenes (for example, comprising lights, and shapes, and colors that move and vary in size, shape, or brightness to correspond with user bio-signals) can also be used. However, it is believed that users—or at least some sub-set of users—will be particularly engaged by interacting with scenes that mimic scenes from the real world (or some fantasy-like rendering of the real world) containing animals, plants, nature landscapes, buildings, wind, weather patterns, bodies of water, sunlight, stars, man-made lighting effects, automobiles, aircraft, and the like. Such scenes are generally referred to herein as “real world scenes,” regardless of whether they may also depict purely fantasy scenes (such as with make-believe animals, or fictitious landscapes or dreamscapes that do not actually exist on Earth). As used herein, a “real world object” or “real world element” is intended to refer to an object or element in a real world scene that depicts an object or element that might exist in that scene, such as a tree in a forest, waves on an ocean, the sound of birds in a field, or the sight of lightening in a cloud. This is as distinguished from purely abstract or incongruent objects or elements, such as Tetris-style blocks.

As an example, a computerized real world scene could be generated to look like a beach, with the waves on the ocean striking the beach in time to the user's breathing, the waves' intensity matched to the user's breathing intensity (as measured by breathing audio level or air volume measurement), an apparent wind sound could also match the user's breathing, the wings of flying seagulls might be matched to the user's heartrate, or the swaying of a palm trees might also match the user's heartrate, while the sunlight glistening on the ocean could create a flowing pattern corresponding with EKG readings.

As another example of a real world scene, a nighttime city street scene could be created wherein the movement of theatrical searchlights across the sky is timed to correspond with the user's breathing, the brightness of the search lights with the breathing intensity, the changing of various building or billboard lights corresponds with the user's heartrate, and the flashing of car lights corresponds with an EKG reading.

As another example of a real world scene, the user might appear to be standing on a wooden ship on the ocean at night, the deck of the ship swaying back and forth in reaction to a rhythmic ocean wave pattern matching the breathing rate of the user, and an aurora borealis moving and varying in size and brightness in a pattern corresponding to the user's heart rate. The sound of a wind can be added matching the user's breathing and the wave movement.

As another example of a real world scene, the user might appear to be under the ocean at a coral reef near a shoreline, with the wave action causing the water to flow back and forth relative to the coral in time to the user's breathing, and fish swimming by with tail movements corresponding to the user's heartrate. Whale song sounds might also correspond to the user's breathing rate, adding an audio element to the bio-feedback. The colors or lighting of the scene might appear to brighten as the user approaches a target breathing and/or heart rate.

The movement, lighting, and sound effects that are synchronized to the user's biometrics may be referred to generally as “biometric synchronized effects.” However, other sounds, objects, movements, and lighting effects can be added as desired. For example, in any of the real world scenes, an overlay of sounds (like whale song, bird and other animal sounds, water sounds, or whatever other background sounds might normally be associated with the scene) may accompany the biometric synchronized effects, but while not being so overwhelming as to make it difficult for the user to readily follow and track the biometric synchronized effects. The “background effects” may add to the realism of the real world scenes for some users, or be otherwise desirable. Music may also be used a background effect.

In general, the real world scenes will make more sense to the user if the movement of the objects in them would otherwise naturally tend to oscillate or vary within the range of the human bio-readings. For example, the movement of the wing of a butterfly, or the lapping of water on a shoreline, or the gait of a large animal, or the movement of a plant in the wind might all believably correspond to the rate of user's breathing or heartrate, making the scenes themselves more believable. Scenes that would otherwise feature faster movement in real life might be run in slower motion to more closely match the rate of user bio signals. Of course, unrealistic rates and speeds of objects in scenes might be employed, though these might be at odds with the expectations of some users seeking a more realistic-looking and realistic-feeling experience.

When integrated into a virtual reality environment, the real world scenes can become an interactive environment that the user's avatar can interact with, walking through the real world scene, looking at it around them in every direction, and seeming to touch and interact with the objects in the scene. For example, in the beach scene described above, the user's avatar could swim in the same ocean whose waves correspond to the user's heartrate. With proper software, a user can be allowed to create their own real world scene to suit their personal tastes and interests, selecting which objects to include, what background effects to add, and which bio-signals control which actions in the scene.

To encourage attainment of a particular breathing rate or heartrate or the like, the biometric audio-visual feedback scenes can change to indicate that a desired condition is being reached. As an example, a real world scene might depict a sailboat on an ocean that is attempting to make headway, wherein the closer the user gets to achieving a desired breathing or heartrate, the faster and more direct the boat's path through the ocean will become, represented by a more consistent wind and steadier rudder. Or in another example, the user's avatar may appear to lift higher off the virtual ground and achieve flight the closer the user gets to reaching desired breathing and heart rates. Other combinations of movements, activities, sounds, and lighting effects may be used for similar purposes.

In some embodiments, multiple users may participate in the same real world scene, with different objects in the scene reflecting biometric data for different users. For example, with reference to the real world scene in FIG. 5, the heart rate of different users can be displayed as differently colored butterflies, while the breathing rate of different users may correspond to the movement of different colored patches of flowers. Color coding (such as, for example, red-colored objects for a first users, blue-colored objects for a second user, and so forth) may help distinguish the objects in the scene.

The biometric audio-visual feedback scenes may be used for a variety of purposes, including mindfulness and meditation, medical observation, health improvement, entertainment, and as a method of creating artistic and creative works (where the scenes are recorded, preserved, and shared).

Certain embodiments of the technology disclosed herein may also be described as follows.

What is claimed is:

1. A device for detecting biometric data, the device comprising:
a necklace having a necklace body configured to be worn around a user's neck;
a plurality of sensors disposed on the necklace body, for detecting biometric data of the user when the necklace is worn around the user's neck;
at least one of an electronic transmitter for transmitting electronic signals corresponding to the biometric data, or an electronic memory for storing biometric data.

2. The device of claim 1, wherein the necklace body has a middle section located to be in alignment with the middle of the users neck and nose when the necklace is worn around the user's neck, wherein the plurality of sensors comprise a first sensor located on the necklace body, one side of the middle section and a second sensor located on the necklace body, on an opposite side of the middle section, and wherein the first and second sensors are separated from each other by the middle section.

3. The device of claim 2, wherein the plurality of sensors include at least one electrocardiogram sensor, electromyogram sensor, or electroencephalogram sensor.

4. The device of claim 2, further comprising a pendant attached to the middle section of the necklace body.

5. The device of claim 4, wherein the plurality of sensors further comprises at least one further sensor on the pendant.

6. The device of claim 1, wherein the plurality of sensors include at least one electrocardiogram sensor, electromyogram sensor, electroencephalogram sensor, microphone sensor or optical sensor.

7. The device of claim 1, further comprising processing electronics on the necklace body and electrically connected to the plurality of sensors for processing sensor signals from the plurality of sensors.

8. The device of claim 7, further comprising at least one power source on the necklace body and electrically connected to the processing electronics to provide electrical power to the processing electronics.

9. The device of claim 1, wherein the plurality of sensors are disposed on the necklace body at positions to contact the user's skin around or under the user's neck, when the necklace is worn around the user's neck.

10. The device of claim 9, wherein the plurality of sensors are configured to detect biometric data through direct contact with the user's skin.

11. The device of claim 1, wherein the body of the necklace further includes a biasing member configured to applying pressure to squeeze said necklace about the user's neck when the necklace is worn around the user's neck.

12. The device of claim 11, wherein the biasing member comprises a semi-rigid, bendable wire along a length dimension of the necklace body.

13. An interactive real-time bio-feedback computer simulation system comprising:
a source of real-time biometric data detected from a user; and
a graphical display depicting a real world scene wherein the movement of a first real world element in the scene corresponds to the user's real-time heartrate, and wherein the movement of a second real world element in the scene corresponds to said user's real-time breathing rate.

14. The system of claim 13, wherein the source of the real-time biometric data comprises the device for detecting biometric data.

15. The system of claim 13, wherein the first real world element comprises an animal or an insect and wherein a movement of the animal or of the insect in said scene corresponds to the user's real-time heartrate.

16. The system of claim 13, wherein the second real world element comprises one or more objects that appear to be moving in the wind, where an amount or a rate of movement of the one or more objects corresponds to said user's real-time breathing rate.
